(19) United States
(12) Patent Application Publication
(10) Pub. No.: US 2014/0188020 A1
(43) Pub. Date: Jul. 3, 2014

(54) Sealskin application system

(71) Applicant: Annamarie Joseph, Charlotte, NC (US)

(72) Inventor: Annamarie Joseph, Charlotte, NC (US)

(21) Appl. No.: 13/731,660

(22) Filed: Dec. 31, 2012

(51) Int. Cl. A61F 15/00

(52) U.S. Cl. CPC A61F 15/004
USPC 602/3

(57) ABSTRACT

A system for providing a protective covering for a human on non-human animal comprising a supply of dispensable plastic stretch film having a predetermined thickness and width and a dispenser-applicator for dispensing a predetermined length of film from the supply thereof and applying the predetermined length of the film to the human or non-human animal.

### BACKGROUND OF THE INVENTION

State-of-the-art medical care requires that open wounds, plaster and other types of casts, porous and non-waterproof bandages and the like require covering by watertight coverings that also prevent access to the injured areas by infectious agents. Thus, the plaster used to form casts around broken or severely injured limbs and joints becomes soft and deteriorates when wet. A patient must make every effort to keep such casts, as well as other types of casts, dry at all times. Moreover, it is necessary to maintain any kind of open wound dry and protected at all times.

In addition, plc lines, incisions, drainage tubes, dressings during bathing, drawing blood (phlebotomy), hygiene, “hot or cold” compresses, numerous treatments or remedies also require protection from infiltration by moisture, water or other fluids.

There have been a large number of prior U.S. patents for covering a cast or sealing the cover for the cast to prevent the intrusion of water into the cast.

U.S. Pat. No. 4,986,265 shows a protective cast cover which covers a cast with a protective plastic cover and wraps the end of the protective cover with a neoprene or foam plastic material to form the seal.

In U.S. Pat. No. 4,911,151, a disposable dressing cover for an arm or a leg provides for a tubular plastic sleeve to fit over the cast and then has one end secured with a strap which is attached by an adhesive.

In U.S. Pat. No. 5,643,183, a waterproof cover for a cast uses a transparent polyethylene bag and seals the distal ends with pairs of strips of hook and loop material.

Similarly, U.S. Pat. No. 4,036,220 has a protective cover and has a flexible collar to form the seal which is attached with hook and loop material.

U.S. Pat. No. 4,229,575 has a flexible bag covering an artificial limb which is sealed with a sealing band.

U.S. Pat. No. 5,342,286 provides a water impervious covering for extremities and uses an adhesive strip in the sealing of a flexible covering.

U.S. Pat. No. 4,363,317 is for a watertight cast cover for protecting a cast which is sealed with a band having overlapping ends forming flaps of hook and loop material.

U.S. Pat. No. 4,610,245 is for a protective sleeve for a human limb which has a sealing cuff which is wrapped tightly around the limb.

U.S. Pat. No. 3,741,203 discloses a protective covering for an injured limb to form a seal with wrapping tape.

In U.S. Pat. No. 5,016,622 an immobilizing orthopedic cast is made of an inner water-impermeable protective sleeve next to the skin and a resilient padding layered with a plastic or resin or glass fiber outer immobilizing layer.

In U.S. Pat. No. 4,523,586 a protective cover for a limb or cast is provided with a sealing band as is U.S. Pat. No. 3,324,580.

In U.S. Pat. No. 5,592,953, a wound cover is formed with a flexible sleeve sealed at each end.

U.S. Pat. No. 4,301,603 concerns a light weight flexible boot worn over a foot in a surgical cast to protect the cast from moisture. The boot can be stretched over various sizes and shapes of casts.

U.S. Pat. No. 4,562,834 relates to a waterproof covering intended to be worn over a cast or bandage on an injured arm or leg comprising a generally tubular water-impervious sleeve which is closed at one end, and which has spaced cutting sites running from side to side along which the sleeve can be cut to different lengths.

U.S. Pat. No. 4,768,501 discloses an air- and water-impervious flexible membrane to be placed over the cast or dressing to a position where the membrane's edge margins overlie the patient's skin along the perimeter of the cast or dressing. A vacuum is created between the membrane and skin by evacuating air through a suction tube or valve.

U.S. Pat. No. 5,016,648 concerns a tubular sleeve of waterproof material for receiving a limb and an elongated closure member for tightening this sleeve around the limb. A portion of the sleeve above the closure member is folded over the closure member to form a cuff. A belt attaches to the cuff and wraps around the cuff to completely seal the covered limb.

U.S. Pat. No. 5,395,302 relates to a limb sheath used to provide a waterproof cover for an injured limb, such as a burned limb or a limb which is bandaged or in a cast. The sheath has a water impervious sleeve made of a first plastics material. The sleeve has an open end through which a limb can be inserted. A sealing strip is fastened to and extends about the internal surface of the sleeve at the open end. The sealing strip is made of a second plastics material which is more highly plasticized than the first plastics material. There is also an elastic strap which is connected to and extends from the sleeve adjacent the open end. The strap is fastened to itself by mating components of a self-contact fastener.

U.S. Pat. No. 5,728,052 concerns a waterproof enclosure including a pliable, waterproof, elongated sheath having either (i) a closed end and an open end, or (ii) two open ends. A resilient, waterproof, sealing element is associated with each open end. A pliable, waterproof, bellows car be interposed between and contiguous with (i) the end of each sealing element and (ii) the sheath. A gas passage through the sheath can be provided that includes a port activated from a first state for inhibiting gas movement through the gas passage and a second state for permitting gas movement through the gas passage.

It is an object of the present invention to provide a novel system for protecting portions of human and non-human animal anatomies requiring medical attention that are much more efficient than those heretofore suggested in the prior art.

### SUMMARY OF THE INVENTION

The above and other objects are realized by the present invention which relates to a system for providing and applying a waterproof, moisture-sealing protective film cover to a portion of the anatomy of a human or non-human animal in need thereof, such as, for example, one bearing a wound, cast, prosthesis, dressing, bandage, compress, drainage tube, or the like.

One of many embodiments of the invention relates to a system for providing a protective covering for at least a portion of a human on non-human animal comprising a supply of dispensable plastic stretch film having a predetermined thickness and width and a dispenser-applicator for dispensing a predetermined length of film from said supply thereof and applying said predetermined length of said film to said human or non-human animal.

An additional embodiment of the invention concerns an article of manufacture comprising packaging material and the above-described system contained within said packaging material, and wherein said packaging material comprises a label which indicates that said system can be used for providing a protective covering for at least a portion of a human on non-human animal.

A still further embodiment of the invention relates to the above-described article of manufacture wherein the packaging material provides protection for the contained system from contamination by water and/or microorganisms.

### BRIEF DESCRIPTION OF THE DRAWINGS

Other objects, features, and advantages of the present invention will be apparent from the written description and the drawings in which:

FIG. 1 shows a perspective view of a dispenser-applicator system in accordance with an embodiment of the invention;

FIG. 2 shows a side view of a dispenser-applicator implemented in the system of FIG. 1.

### DETAILED DESCRIPTION OF THE PREFERRED EMBODIMENTS

A more complete understanding of the present invention may be derived by referring to the detailed description and claims when considered in connection with the Figures, wherein like reference numbers refer to similar items throughout the Figures.

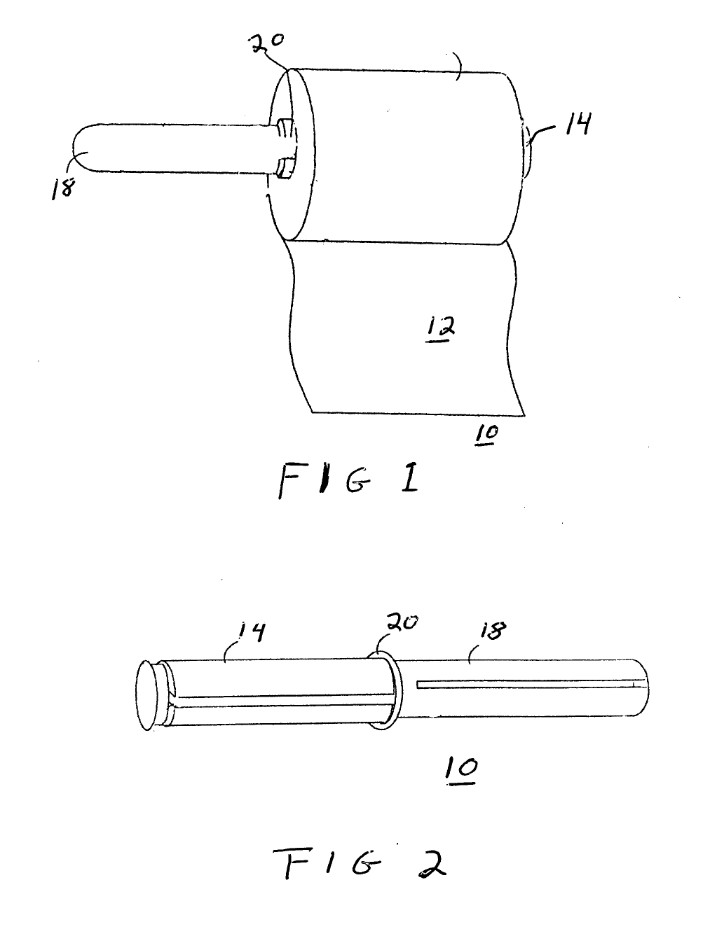

Referring to FIGS. 1-2, FIG. 1 shows a perspective view of a dispenser-applicator system 10 in accordance with an embodiment of the invention. FIG. 2 shows a side view of a specific dispenser-applicator implemented in system 10.

Referring specifically to FIG. 1, a supply of plastic stretch film 12 is pre-wound onto spool 14 of a dispenser-applicator 16. Applicator 16 also includes a preferably ergonomic handle 18. Dispenser-applicator 16 is optionally coupled to and longitudinally aligned with telescoping mechanism 20 which allows, if desired, extending the spool 14 to enable application of the film 12 to hard-to-reach areas.

FIG. 2 shows a side view of dispenser applicator 16 without the pre-wound plastic stretch film so as to further illustrate the telescoping mechanism 20.

Although the preferred embodiments of the invention have been illustrated and described in detail, it will be readily apparent to those skilled in the art that various modifications may be made therein without departing from the spirit of the invention or from the scope of the appended claims.

For example, the system may be designed for single usage applications or for situations where multiple lengths of film are separately dispensed for applications to multiple sites; either on the same or different patients. Moreover, the film may assume any convenient shape, design or construction. For example, the film may be perforated, it being understood that the system is designed for multiple wrappings of the perforated sheet around the designated site so that the perforations overlap with non-perforated sections to provide a waterproof and protective covering.

I claim:

1. A system for providing a protective covering for at least a portion of a human on non-human animal comprising a supply of dispensable plastic stretch film having a predetermined thickness and width and a dispenser-applicator for dispensing a predetermined length of film from said supply thereof and applying said predetermined length of said film to said human or non-human animal.

2. The system of claim 1 wherein said supply of plastic stretch film comprises a roll of said film.

3. The system of claim 2 wherein said dispenser-applicator is optionally coupled to and longitudinally aligned with a telescoping mechanism which enables extension of the roll of said film.

4. The system of claim 1 wherein said plastic stretch film is substantially transparent.

5. The system of claim 1 wherein said plastic stretch film is colored.

6. The system of claim 6 wherein said plastic stretch film is a skin tone color.

7. The system of claim 1 wherein said plastic stretch film comprises a polyolefin, polyvinyl chloride or ethylene vinyl acetate.

8. The system of claim 7 wherein said polyolefin comprises polyethylene, polypropylene or polybutylene.

9. An article of manufacture comprising packaging material and the system of claim 1 contained within said packaging material, and wherein said packaging material comprises a label which indicates that said system can be used for providing a protective covering for at least a portion of a human on non-human animal.

10. The article of manufacture of claim 9 wherein said packaging material provides protection for said contained system from contamination by water and/or microorganisms.
